(19) United States
(12) Patent Application Publication
(10) Pub. No.: US 2008/0317199 A1
(43) Pub. Date: Dec. 25, 2008

(54) Medical Diagnostic Apparatus Comprising a Drive System for Positioning a Radiation Source Facing a Radiation Detector

(75) Inventor: Adrianus Van Der Ende, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONIC, N.V., EINDHOVEN (NL)

(21) Appl. No.: 10/596,413

(22) PCT Filed: Dec. 13, 2004
(86) PCT No.: PCT/IB2004/052790

§ 371 Date: Jun. 13, 2006

(30) Foreign Application Priority Data
Dec. 16, 2003 (EP) 03104712.9

(51) Int. Cl. A61B 6/00

(52) U.S. Cl. 378/19

(57) ABSTRACT

The invention relates to a medical diagnostic device comprising a drive system (15) for positioning a radiation source (3) and a radiation detector (5) facing each other with respect to a target (9). The medical diagnostic device comprises a frame (11) and a support (1). Via a central position the support (1) can be rotated with respect to the frame (11) between a first and a second extreme position. The drive system comprises a first (19) and a second (21) guiding wheel. At least one of the guiding wheels (19, 21) is driven by a drive means (27). Furthermore the drive system (15) comprises a belt (17) which is connected to the support (1) by two connection points (23, 25). In the central position of the support (1) the belt (17) is guided over and along the first (19) and the second (21) guiding wheels so that a zigzag configuration of the belt (17) is obtained. Due to the zigzag configuration of the belt (17) at least one of the connection points (23, 25) of the belt can move along its corresponding guiding wheel (19,21) providing a larger angular range than the angular range in similar constructions.

The invention relates to a medical diagnostic apparatus comprising a drive system for positioning a radiation source facing a radiation detector relative to a target object, the apparatus comprising a frame and a support for the radiation source and the radiation detector, which support is bearing-mounted and rotatable with respect to the frame, the support via a central position relative to the frame being rotatable between a first and a second extreme position.

A medical diagnostic apparatus of the type defined in the opening paragraph is known from U.S. Pat. No. 4,955,046. The known medical diagnostic apparatus is an X-ray apparatus and comprises a C-arc construction, which is rotatable by means of a timing belt which is led over a drivable cogwheel. An X-ray radiation source and an X-ray detector are attached to the C-arc construction, diametrically relative to each other. Also an additional drive pulley is used which provides that the contact face between the timing belt and the drivable cogwheel is enlarged, so that the chance of slip between the belt and the cogwheel is reduced.

For example for 3D-image reconstructions by means of such an X-ray apparatus a largest possible angular range is of importance. In general it holds that the larger the angular range of the C-arc construction, the better the quality of the 3D-image reconstructions. The angular range of the C-arc construction of the known X-ray apparatus is determined by the angular distance between the first and the second connection point of the belt to the C-arc and cannot exceed this angular distance.

It is an object of the invention to provide a medical diagnostic apparatus of the type defined in the opening paragraph, which comprises a rotatable support that has a larger angular range than the C-arc construction of the known medical diagnostic apparatus.

The intended object is achieved by a medical diagnostic apparatus according to the invention, characterized in that the drive system comprises first and second timing pulleys, at least one of the first and second timing pulleys being drivable by a drive element and the drive system further comprising a belt which is attached to the support via a first and a second connection point, wherein, at any rate in central position of the support, the belt runs from the first connection point in a zigzag configuration over the first and second timing pulleys to the second connection point. Since with a zigzag configuration of the belt it is possible to come with at least one of the connection points past one of the first and second timing pulleys when the support is rotated relative to the frame, the angular range of the rotatable support in the medical diagnostic apparatus according to the invention is not restricted to a maximum of the angular distance between the first and the second connection point from the belt to the support. This provides an angular range that exceeds the angular range of the C-arc construction of the known medical diagnostic apparatus. Belt within the context of this application is understood to mean for example a belt or a timing belt, a chain, a band or a V-belt.

A practical embodiment of the zigzag configuration of the belt is achieved in an embodiment of a medical diagnostic apparatus according to the invention, characterized in that in the central position of the support the zigzag configuration of the belt is present because the belt runs from the first connection point along the first timing pulley, over the second timing pulley, over the first timing pulley and along the second timing pulley to the second connection point, in this order, at any rate, wherein, in the central position of the support, the first timing pulley is positioned in closer proximity of the first connection point than of the second connection point and the second timing pulley is positioned in closer proximity of the second connection point than of the first connection point. As a result of the zigzag configuration of the belt, parts of the belt lie side by side over the support when the support is in central position. Another part of the belt is preferably free from the support.

One embodiment of the invention is a medical diagnostic apparatus, characterized in that the part of the belt running from the second timing pulley to the first timing pulley is led over a third timing pulley of the drive system, the third timing pulley being located between the first and the second timing pulley and between the support and the belt. The advantage of this embodiment is that the distance between the first and the second timing pulley can be enlarged, as a result of which the angular range of the rotatable support increases. Another advantage of this embodiment is that the part of the belt that is preferably free from the support is kept at some distance from the support by the third timing pulley, so that this part of the belt is led along the support so that the drive of the support is improved compared to when this part too would run over the support.

One embodiment of the invention is a medical diagnostic apparatus, characterized in that the support comprises a C-arc to which the radiation source and the radiation detector are attached diametrically relative to each other and in which the radiation source and the radiation detector comprise an X-ray radiation source and an X-ray detector.

It is observed that from DE-4214087 is known a medical diagnostic apparatus for positioning a radiation source facing a radiation detector relative to a target object. This known medical diagnostic apparatus comprises a double C-arc construction with a first and a second C-arc, the first C-arc comprising a radiation source and radiation detector installed diametrically relative to each other. The first C-arc is connected to the second C-arc by means of a holder so that, for positioning the radiation source and the radiation detector relative to a target object, an angular range is possible that is larger than in the case of the known single C-arc constructions. However, the double C-arc construction is disadvantageous in that the construction is more expensive, bulkier and heavier than the single C-arc construction. The use of the construction according to the invention is advantageous in that it is possible in a simple manner to reconstruct 3D-images better than possible thus far by means of relatively cost-effective, compact and light equipment.

Embodiments of a medical diagnostic apparatus according to the invention will be explained in more detail with reference to the following Figures in which:

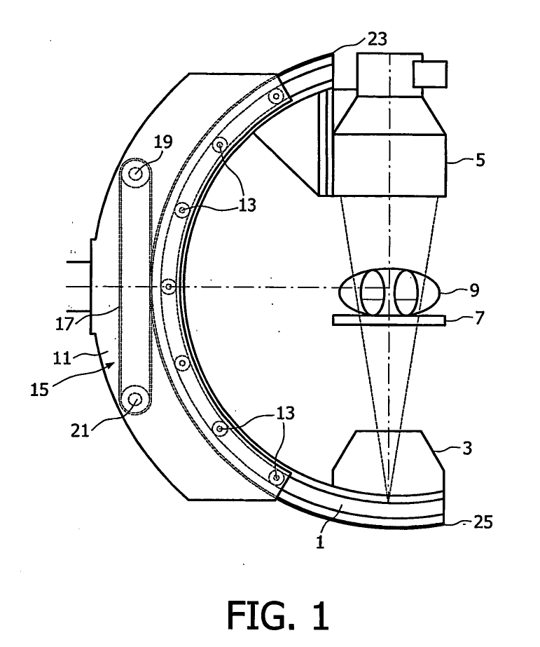

FIG. 1 is a diagrammatic front elevation of a first embodiment of a medical diagnostic apparatus according to the invention,

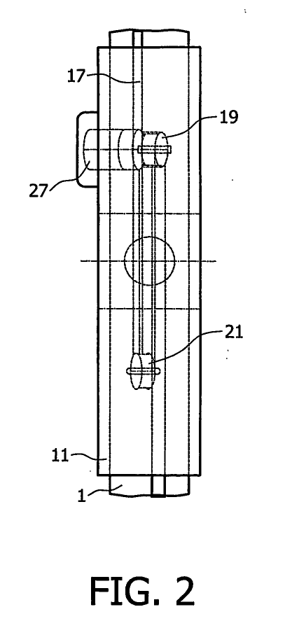

FIG. 2 is a diagrammatic side elevation of a part of a medical diagnostic apparatus as shown in FIG. 1,

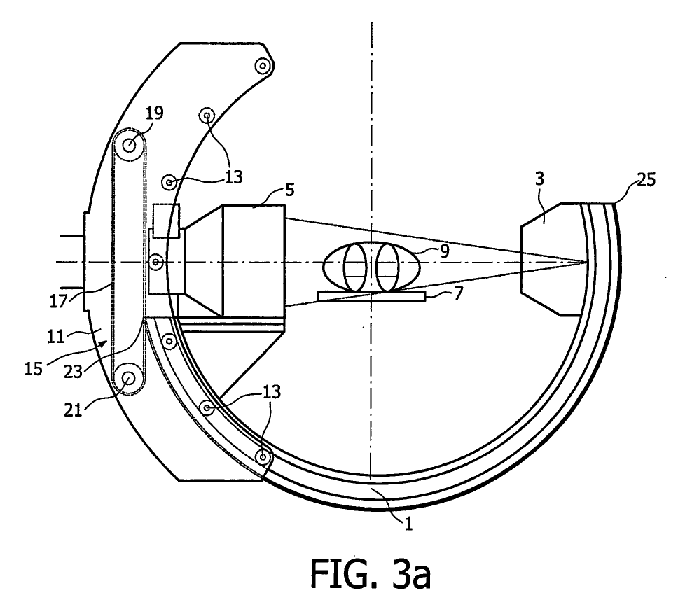

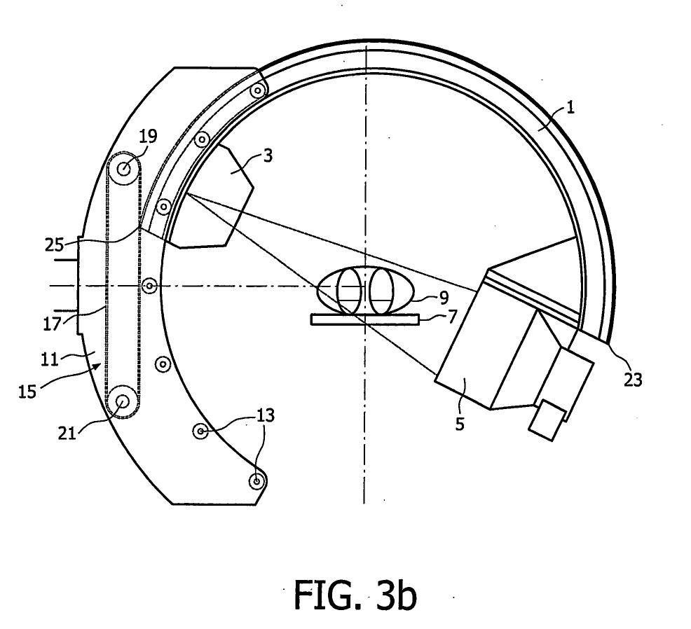

FIG. 3 is a diagrammatic representation of one of two possible extreme positions of the medical diagnostic apparatus shown in FIG. 1,

FIG. 3b is a diagrammatic representation of the other possible extreme position of the medical diagnostic apparatus shown in FIG. 1,

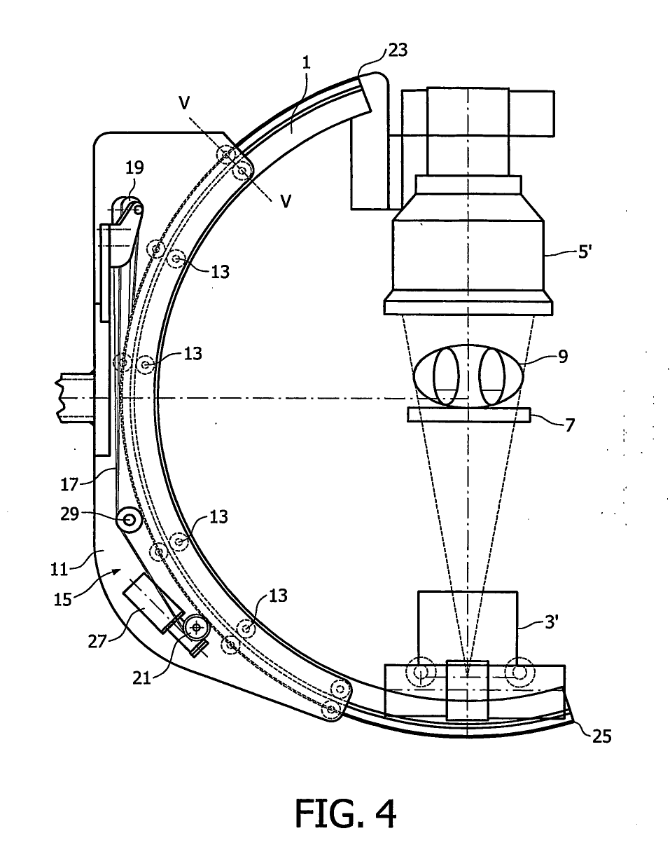

FIG. 4 is a diagrammatic representation of a second embodiment of a medical diagnostic apparatus according to the invention, and

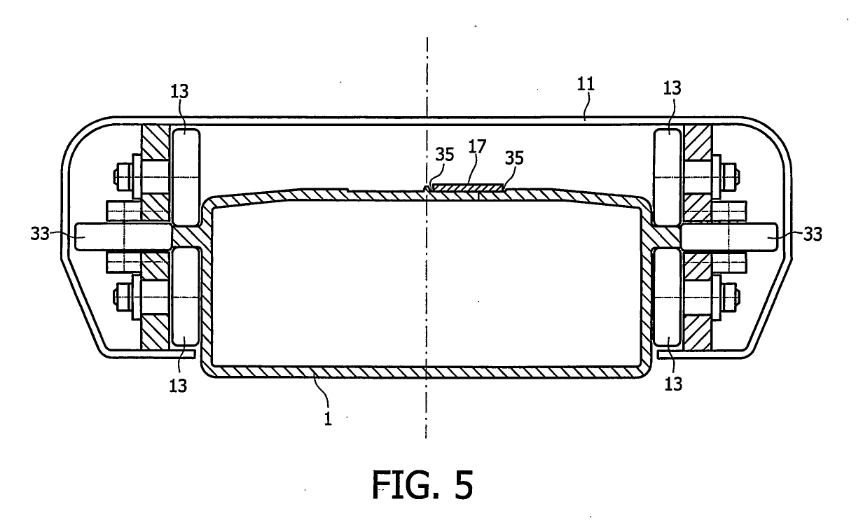

FIG. 5 shows a cross section along the line V-V in FIG. 4.

FIG. 1 gives a diagrammatic representation of part of a medical diagnostic apparatus according to the invention. In the embodiment shown the medical diagnostic apparatus is an X-ray apparatus. The X-ray apparatus comprises a C-arc 1 extending at an approximately 180° angle with attached thereto, diametrically to each other, an X-ray radiation source 3 and an X-ray detector 5. By rotation of the C-arc 1 the X-ray radiation source 3 and the X-ray detector 5 can be positioned relative to a patient table 7 and a patient 9. The C-arc 1 is bearing-mounted and rotatable relative to a frame 11. By means of an array of guide rollers 13 and a drive system 15 the C-arc 1 can be rotated relative to the frame. The drive system 15 comprises a belt 17, a first timing pulley 19, a second timing pulley 21 and at least one drive element, which is not shown in this Figure. The belt 17 is attached to the respective extreme positions of the C-arc 1 by means of a first connection point 23 and a second connection point 25. The first and second timing pulleys 19 and 21 are bearing-mounted and rotatable relative to the frame 11 and are positioned in close proximity of the outer circumference of the C-arc 1, the first timing pulley being located in the central position of the C-arc shown in FIG. 1, in closer proximity of the connection point 23 than of the second connection point 25 and the second timing pulley in FIG. 1 being located in closer proximity of the second connection point than of the first connection point. The belt 17 runs from the first connection point 23 first along the first timing pulley 19 and then over the second timing pulley 21 and over the first timing pulley 19, in this order. Then the belt 17 runs along the second timing pulley 21 to the second connection point 25. By having the belt 17 run between the two connection points 23 and 25 along and over the first and second timing pulleys 19 and 21, a zigzag configuration of the belt is created. This enables the connection points 23 and 25 to come past the first and second timing pulley 19 and 21 respectively when the C-arc 1 is rotated, so that a larger angular range is obtained than with prior-art C-arc constructions, such as known from U.S. Pat. No. 4,955,046.

A consequence of this drive system 15 in which the belt 17 runs via a zigzag configuration over the first and second timing pulleys 19 and 21 is that a longer belt is required than with the known drive systems for C-arc constructions, so that stringent requirements are to be set to the elasticity of the belt. For that matter it holds in general that the movement of a system driven by a belt is better controllable and reproducible as the elasticity of the belt is smaller. In order to realize a movement of the C-arc that can be controlled and reproduced in the best possible way, preferably a timing belt having a smallest possible elasticity is used in combination with first and second timing pulleys. Needless to observe that also other embodiments of a belt and associated timing pulleys can be used such as, for example, a chain, a band or even a V-belt with associated timing pulleys.

FIG. 2 gives a diagrammatic representation of a possible positioning of the first and second timing pulleys 19 and 21, the timing pulleys being positioned slightly rotated and translated relative to each other and the first timing pulley 19 being connected to a drive element 27, in the embodiment shown a conventional electromotor. The zigzag configuration of the belt 17 between the first connection point 23 and the second connection point 25 along and over the first and second timing pulleys 19 and 21 is clearly visible in this figure. As a result of the combination of a zigzag configuration of the belt 17 and a positioning of the first and second timing pulleys 19 and 21 slightly rotated and translated relative to each other, it is possible in the central position of the C-arc 1 to have a part of the belt that is located between the first connection point 23 and the second timing pulley 21 and a part of the belt that is located between the first timing pulley 19 and the second connection point 25 run side by side along the outer circumference of the C-arc. For, by positioning the first and second timing pulleys 19 and 21 slightly rotated and translated relative to each other, the belt 17, seen from the first connection point 23, is simply led from the second timing pulley 21 to the first timing pulley 19 without parts of the belt running against the side of one of the timing pulleys, and parts of the belt ending up across each other along the outer circumference of the C-arc 1. As a result, unnecessary friction and wear of the belt 17 is avoided, so that the life of the belt is extended.

The FIGS. 3a and 3b both give a diagrammatic representation of an extreme position of the part of the X-ray apparatus shown in FIG. 1. In FIG. 3a the C-arc 1 is rotated such that the X-ray source 3 is located on the side of the patient table 7 with the patient 9. Because of this rotation of the C-arc 1 the first connection point 23 of the belt 17 has passed the first timing pulley 19 and the first connection point 23 is even in close proximity of the second timing pulley 21. Similar to this, FIG. 3b shows the other extreme position of the part of the X-ray apparatus shown in FIG. 1, in which the C-arc 1 is turned the other way than in FIG. 3a. In FIG. 3b the second connection point 25 of the belt 17 has passed the second timing pulley 21 and the second connection point 25 is in close proximity of the first timing pulley 19. The extreme positions of the part of the X-ray apparatus shown in FIG. 1, which are shown in FIGS. 3a and 3b, determine the maximum angular range of the C-arc 1 of the X-ray apparatus. This maximum angular range is considerably larger than the angle at which the C-arc extends.

It will be obvious to the expert that the part of the C-arc 1 that is still within the frame 11 when the C-arc has reached its extreme positions, should be sufficiently large to guarantee the stability of the X-ray apparatus.

FIG. 4 gives a diagrammatic representation in front elevation of part of a second embodiment of a medical diagnostic apparatus according to the invention; an X-ray apparatus in the example shown. The X-ray apparatus comprises, just like FIG. 1, a bearing-mounted C-arc 1, which arc is rotatable relative to a frame 11 by means of a system of guide rollers 13 with, connected to this C-arc diametrically relative to each other, an X-ray source 3′ and an X-ray detector 5′ which can be positioned relative to a patient table 7 and a patient 9 by rotation of the C-arc. Again the C-arc 1 can be rotated relative to the frame 11 by means of a drive system 15. Besides a belt 17, which is connected to the respective outer ends of the C-arc 1 by a first and a second connection point 23 and 25, first and second timing pulleys 19 and 21 and a drive element 27 which drives the second timing pulley 21, the drive system 15 comprises also a third timing pulley 29. The third timing pulley 29 is located in close proximity of the outer circumference of the C-arc 1, between the first and second timing pulleys 19 and 21 and between the C-arc 1 and the belt 17 and is bearing-mounted and rotatable relative to the frame 11 just like the first and second timing pulleys. The zigzag configuration of the belt 17 is obtained by the belt running from the first connection point 23 to the second connection point 25 along the first timing pulley 19, over the second timing pulley 21, over the third timing pulley 29, over the first timing pulley 19 and along the second timing pulley 21, in this order.

A considerable advantage of including the third timing pulley 29 in this example of embodiment is that as a result of this third timing pulley the first and second timing pulleys 19 and 21 can be placed further apart, realizing a larger angular range. Another advantage of including the third timing pulley 29 in this example of embodiment is that the third timing pulley keeps the part of the belt 17, positioned between the second timing pulley 21 and the first timing pulley 19, clear of the C-arc 1, so that this part of the belt is properly led along the C-arc and is avoided ending up across other parts of the belt over the C-arc. This provides a better drive of the C-arc 1 than in the case where there is no third timing pulley 29.

Although for the embodiment shown in FIG. 4 three timing pulleys 19, 21 and 29 are sufficient for optimum functioning of the drive system 15, constructions are conceivable in which the addition of extra timing pulleys further improves the drive system. However, the function and position of these extra timing pulleys correspond to those of the third timing pulley 29.

As is clearly visible in FIG. 4 the first timing pulley 19 and the second timing pulley 21 are positioned rotated and translated relative to each other. This positioning of the first and second timing pulleys 19 and 21 provides that, seen from the first connection point 23, the belt 17 is led as it were automatically from the second timing pulley, along the third timing pulley 29 over the first timing pulley, without parts of the belt running against the side of one of the timing pulleys and parts of the belt ending up across each other along the outer circumference of the C-arc 1.

FIG. 5 shows a cross section of the bearing of the C-arc 1 of the X-ray apparatus along the line V-V in FIG. 4. The C-arc is partly countersunk in the frame 11 and is bearing-mounted and rotatable relative to the frame by means of the system of guide rollers 13. The C-arc 1 is positioned in axial direction relative to the frame by means of a system of positioning rollers 33. Both the guide rollers 13 and the positioning rollers 33 are bearing mounted and rotatable relative to the frame 11. Two parallel grooves 35 are provided along the outer circumference of the C-arc 1, so that the parts of the belt 17 which, due to the zigzag configuration of the belt, can end up side by side along part of the outer circumference of the C-arc, are led through the separate grooves on the C-arc. This avoids parts of the belt 17 shifting along the C-arc 1, by which the functioning of the drive system 15 could be adversely affected. A comparison with FIG. 4 shows that in the part of the C-arc 1 shown in FIG. 5, only one part of the belt 17 runs along the outer circumference of the C-arc. This part of the belt runs through only one of the grooves 35. The other groove 35 in the respective part of the C-arc 1 is empty.

1. A medical diagnostic apparatus comprising a drive system (15) for positioning a radiation source (3) facing a radiation detector (5) relative to a target object (9), the apparatus comprising a frame (11) and a support (1) for the radiation source (3) and the radiation detector (5), which support is bearing-mounted and rotatable with respect to the frame (11), the support via a central position relative to the frame (11) being rotatable between a first and a second extreme position, characterized in that the drive system (15) comprises first and second timing pulleys (19, 21), at least one of the first and second timing pulleys (19, 21) being drivable by a drive element (27) and the drive system (15) further comprising a belt (17) which is attached to the support (1) via a first and a second connection point (23, 25), wherein, at any rate in central position of the support (1), the belt (17) runs from the first connection point (23) in a zigzag configuration over the first and second timing pulleys (19, 21) to the second connection point (25).

2. A medical diagnostic apparatus as claimed in claim 1, characterized in that in the central position of the support (1) the zigzag configuration of the belt (17) is present because the belt (17) runs from the first connection point (23) along the first timing pulley (19), over the second timing pulley (21), over the first timing pulley (19) and along the second timing pulley (21) to the second connection point (25), in this order, at any rate, wherein, in the central position of the support (1), the first timing pulley (19) is positioned in closer proximity of the first connection point (23) than of the second connection point (25) and the second timing pulley (21) is positioned in closer proximity of the second connection point (25) than of the first connection point (23).

3. A medical diagnostic apparatus as claimed in claim 1, characterized in that the part of the belt (17) running from the second timing pulley (21) to the first timing pulley (19) is led over a third timing pulley (29) of the drive system (15), the third timing pulley (29) being located between the first and the second timing pulleys (19, 21) and between the support (1) and the belt (17).

4. A medical diagnostic apparatus as claimed in claim 1, characterized in that the support (1) comprises a C-arc to which the radiation source (3) and the radiation detector (5) are attached diametrically relative to each other and in which the radiation source (3) and the radiation detector (5) comprise an X-ray radiation source and an X-ray detector.
